(19) United States
(12) Patent Application Publication
(10) Pub. No.: US 2026/0053539 A1
(43) Pub. Date: Feb. 26, 2026

(54) Screw Assembly

(71) Applicant: Stryker European Operations Limited, Carrigtwohill (IE)

(72) Inventor: Joël Bouquet, Lommiswil (CH)

(21) Appl. No.: 19/297,388

(22) Filed: Aug. 12, 2025

Related U.S. Application Data
(60) Provisional application No. 63/685,418, filed on Aug. 21, 2024.

(51) Int. Cl. A61B 17/86; A61B 17/72

(52) U.S. Cl. CPC A61B 17/864; A61B 17/7225; A61B 17/8615; A61B 17/863

(57) ABSTRACT

A screw assembly and a method of inserting the assembly into a bone are described herein. The screw assembly includes a cannulated screw having a first thread with a first handedness and a blocking screw with a second thread having an opposite handedness creating a cross-working thread assembly.

### CROSS-REFERENCE TO RELATED APPLICATIONS

This application claims the benefit of the filing date of U.S. Provisional Ser. No. 63/685,418, filed Aug. 21, 2024, the disclosure of which is hereby incorporated herein by reference.

### BACKGROUND OF THE INVENTION

The present application relates to devices and methods for fixating a fractured bone using a screw assembly, for instance, fixing fractures in a metacarpal.

Bone fractures are often treated by fixing two or more bone fragments through the use of bone plate and screw constructs or other types of fixation devices, such as threaded or unthreaded nails. In general, these fracture fixation devices engage the fragments to stabilize and compress the fractured bone fragments and thus facilitate the healing process. For instance, in the case of intramedullary screws, the threading of the component into the bone often acts to compress and fix the fragments concurrently.

Metacarpal fractures often require the implantation of fixation devices to align and facilitate fracture healing. Intramedullary screws for fixation of bones are a rising method for securing metacarpal fractures as the device allows for a greater range of motion, faster recovery, limited dissection, and limited complications. However, when complications do arise, they often relate to malunion and nonunion of the fractured bones. These complications may arise after the setting of a fixation device and can be related to the method of fixation.

For example, fractures near the joint present an issue for fixation devices. In the proximal metacarpal bone, there is a large amount of cancellous bone near the joint which creates a possibility for screws to become loose or even in some cases fall out resulting in the aforementioned malunion or nonunion. Metacarpal bones see a substantial amount of movement making loosening a substantial issue for intramedullary screws, which are not completely rotationally stable meaning that movement may result in the unwanted loosening of the screw. Unfortunately, fixation devices that are more rotationally stable such as plates do not provide the same range of motion, limited dissection, and recovery rate compared to intramedullary screws making them an inadequate alternative.

Therefore, there exists a need for an intramedullary screw that does not suffer from unwanted loosening and resultant bone nonunion.

### BRIEF SUMMARY OF THE INVENTION

In an embodiment the screw assembly is a device for fixating a fracture using an screw combination that facilitates the healing of the bone by rotationally stabilizing and compressing fracture segments. The assembly has a screw that features a hollow inner portion which has been configured to receive a second screw. When the second screw is placed within the first screw an interweaving thread pattern is created which locks the screws into position.

The screw assembly is inserted in a two-step process. A first hollow screw is inserted into a intramedullary portion of a bone (e.g., a metacarpal). The first screw is rotated into two fracture fragments holding the bone together. A second screw is then inserted into the first with a different thread orientation which causes the fractured bone to compress. The threads intersect creating a interwoven pattern that locks the first screw into place preventing rotation. As a result of the interwoven pattern the screw assembly becomes rotationally and translationally stabilized thus reducing the chance of the assembly coming loose in the bone. As a result, there is a lower probability of nonunion occurring which would require additional procedures for corrections. The different thread orientations of the screw components (e.g., left vs. right-handed) may require the screws to be rotated into place in different rotational orientations (e.g., clockwise vs. counterclockwise).

Another embodiment is a first screw assembly may include a cannulated screw including a first thread having a first handedness and a blocking screw including a second thread having a second handedness opposite of the first handedness. When the blocking screw is inserted within the cannulated screw a portion of the second thread extends out of a portion of the cannulated screw.

In the foregoing embodiment, the cannulated screw may include a thread cutout for receiving a portion of the second thread. The first and second threads may have different pitches. The first thread may have at least two different pitches. The first thread may have a first threaded section and a second threaded section having different pitches. A major diameter of the blocking screw may be greater than a minor diameter of the cannulated screw. The first handedness may be right hand oriented, and the second handedness may be left hand oriented, or vice versa. The blocking screw may include a head at a proximal end with a drive shape that is one of a slotted, a cruciform, a square, a multiple-square, a internal hex, a pentalobular, a hexalobular, a combination, a external, or a tamper resistant shape. The cannulated screw may include a head at a proximal portion of the cannulated screw, and that head may have a drive shape that is one of a slotted, a cruciform, a square, a multiple-square, a internal hex, a pentalobular, a hexalobular, a combination, a external, or a tamper resistant shape. The cannulated screw may include a thread cutout that extends through the head of the cannulated screw.

Yet another screw assembly may include a cannulated screw including a first thread having a first handedness and a thread cutout extending a length of the cannulated screw having a helical opening between a cavity and an exterior of the cannulated screw and a blocking screw including a second thread having a second handedness opposite of the first handedness. When the blocking screw is inserted within the cannulated screw a portion of the second thread extends partially out of the thread cutout and engages a bone.

A further embodiment is a method of repairing a fracture in a bone that may include rotating a cannulated screw in a first direction into the bone and rotating a blocking screw in a second direction into the cannulated screw such that at least a portion of the blocking screw engages the bone.

The foregoing method may further include rotating the cannulated screw in a clockwise direction and rotating the blocking screw includes rotating in a counter-clockwise direction. The method may also include rotating the cannulated screw in a counter-clockwise direction and rotating the blocking screw includes rotating in a clockwise direction. Rotating the blocking screw may include permitting the portion of the blocking screw through a thread cutout in the cannulated screw. The method may also include drilling a hole into the bone to guide the insertion of the cannulated screw and/or inserting a guide wire into the hole to guide the insertion of the cannulated screw. The method may also include counterboring an entry portal into the bone before inserting the cannulated screw.

### BRIEF DESCRIPTION OF THE DRAWINGS

FIG. 1A is a perspective view of a screw assembly according to an embodiment of the present disclosure.

FIG. 1B is a side view of a screw assembly of FIG. 1A.

FIG. 1C is a view of the distal end of the screw assembly of FIG. 1A.

FIG. 1D is a view of the proximal end of the screw assembly of FIG. 1A.

FIG. 2A is a perspective view of a cannulated screw of the screw assembly of FIG. 1A.

FIG. 2B is a side view of the cannulated screw of FIG. 2A.

FIG. 2C is a view of the distal end of the cannulated screw of FIG. 2A.

FIG. 2D is a view of the proximal end of the cannulated screw of FIG. 2A.

FIG. 3A is a perspective view of a blocking screw of the screw assembly of FIG. 1A.

FIG. 3B is a side view of the blocking screw of FIG. 3A.

FIG. 3C is a view of the distal end of the blocking screw of FIG. 3A.

FIG. 3D is a view of the proximal end of the blocking screw of FIG. 3A.

FIG. 4 is a cross sectional view along cross section A-A of the screw assembly of FIG. 1B.

FIG. 5 illustrates a partially transparent bone with a fracture and predrilled and counterbored hole for receiving the screw assembly of FIG. 1A.

FIG. 6 illustrates the cannulated screw of FIG. 2A partially placed within the hole of FIG. 5.

FIG. 7A illustrates the cannulated screw of FIG. 2A fully inserted into the hole of FIG. 5.

FIG. 7B is a proximal end view of the cannulated screw of FIG. 7A.

FIG. 8 illustrates the blocking screw of FIG. 3A partially placed within the cannulated screw of FIG. 7A.

FIG. 9A illustrates the blocking screw of FIG. 3A fully inserted into the cannulated screw of FIG. 7A.

FIG. 9B is a proximal end view of the blocking screw of FIG. 9A.

### DETAILED DESCRIPTION

As used herein unless stated otherwise, the term “anterior” means toward the front part of the body, and the term “posterior” means toward the back part of the body. When referring to specific directions in the following discussion of a certain device, the terms “proximal” and “distal” are to be understood in regard to the device's orientation and position during exemplary application to human body. Thus, the term “proximal” means closer to the operator or in a direction toward the operator, and term “distal” means more distant from the operator or in a direction away from the operator. In addition, the terms “about,” “generally,” and “substantially” are intended to mean that deviations from absolute are included within the scope of the term so modified.

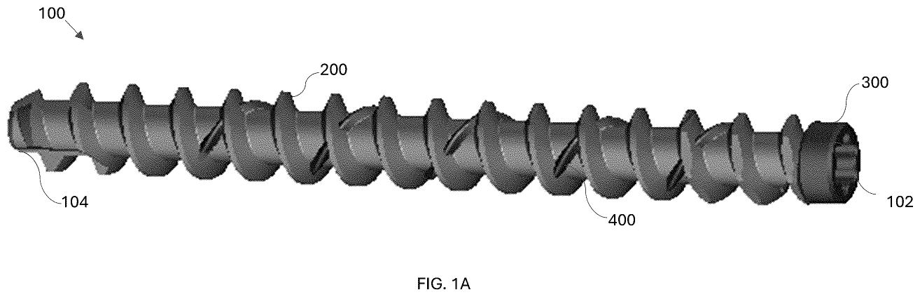

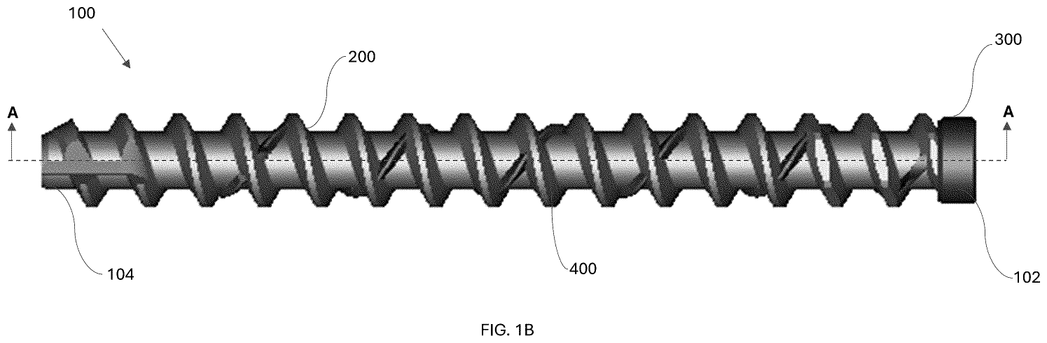

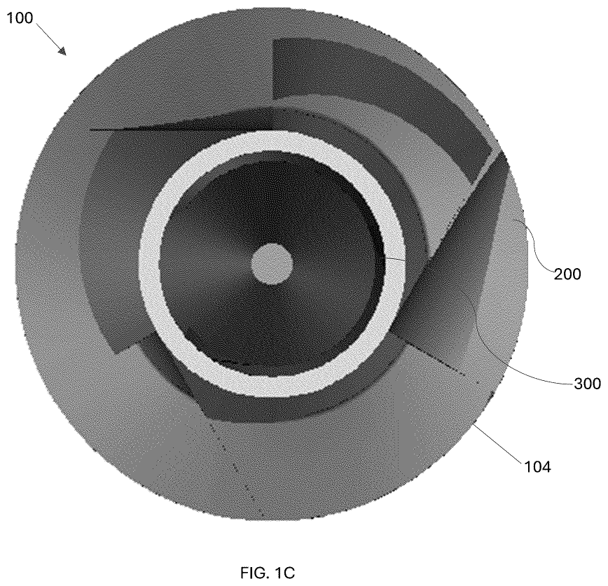

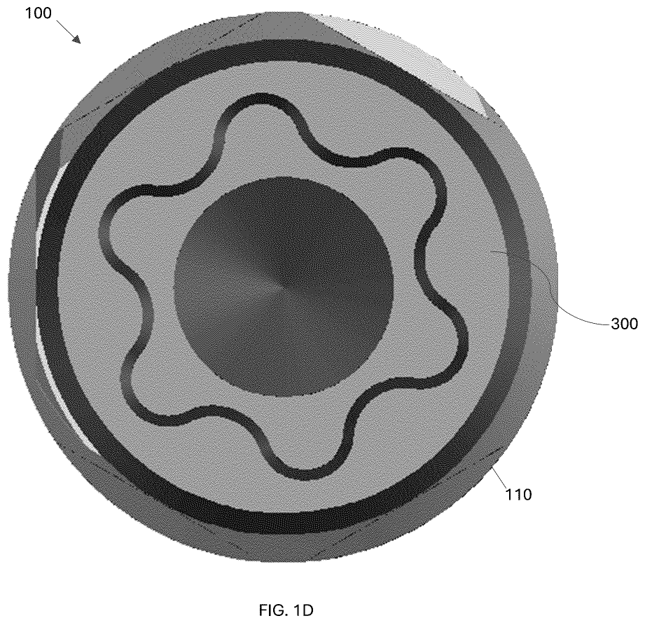

As shown in FIGS. 1A-1D, screw assembly 100 is designed for fixing a fractured bone such that the fracture fragments are stabilized and compressed. While discussed herein largely in connection with the fixation of a metacarpal, assembly 100 can be tailored for use in connection with other bones, such as phalanges, radius, ulna, humerus, tibia, fibula, femur, metatarsal or the like. Assembly 100 includes cannulated screw 200 and blocking screw 300. These individual components are discussed more fully below, but each have exterior threading exhibiting opposite handedness and each cooperate with the other to create a cross-working thread assembly 400. Cross-working thread assembly 400 engages the bone and acts to prevent screw assembly 100 from loosening due to phenomena such as vibrations while also minimizing patient irritation. As shown, screw assembly 100 includes a proximal end 102 and a distal end 104.

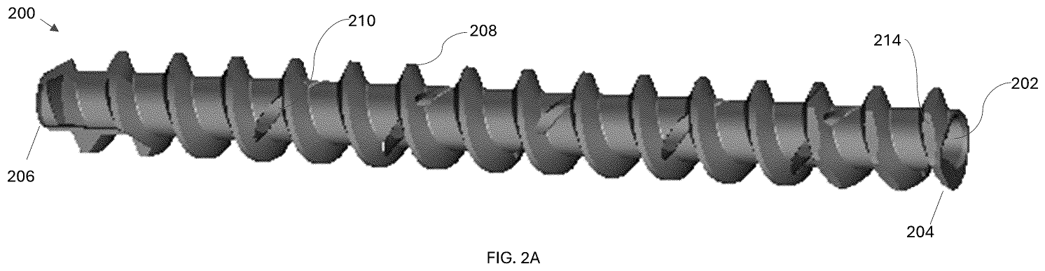

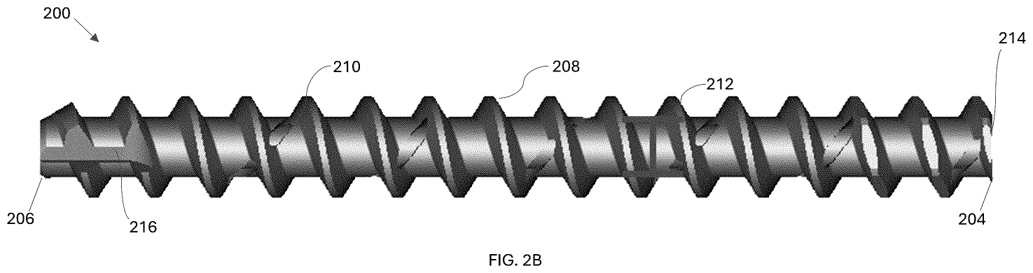

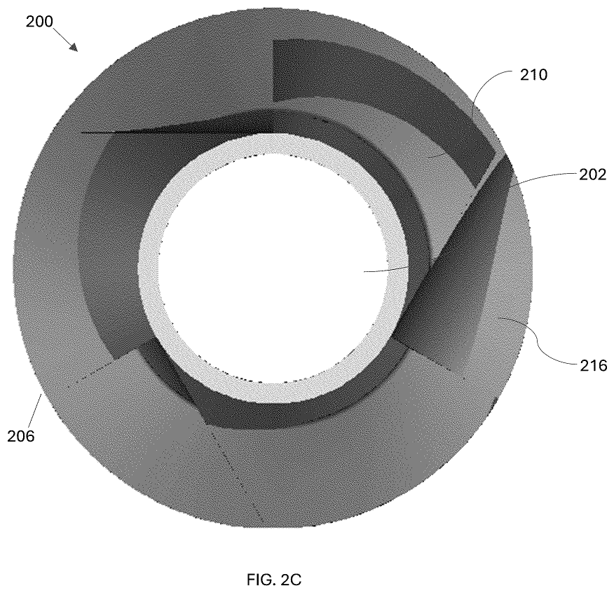

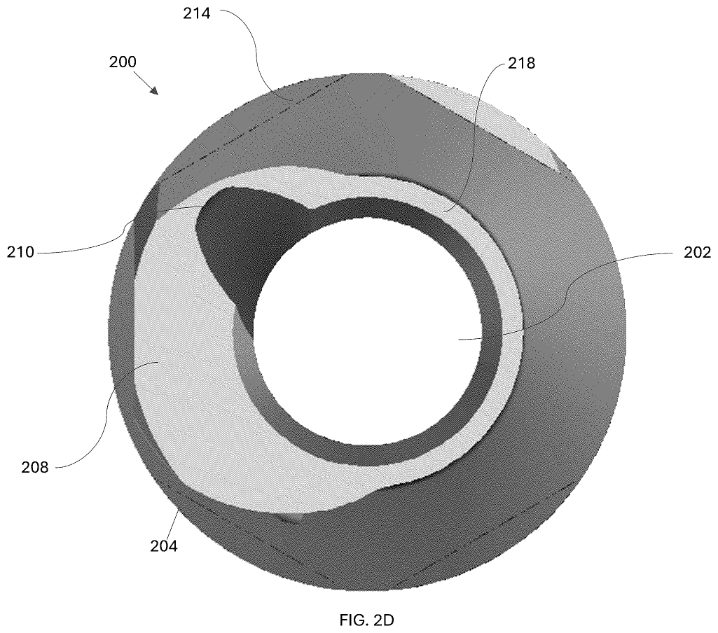

As shown in FIGS. 2A-2D, cannulated screw 200 includes a cannulation 202 that runs along the length of the screw and is configured to receive blocking screw 300 (discussed below) from the proximal end 204 towards the distal end 206, as is discussed more fully below. An exterior of screw 200 includes a first thread 208 exhibiting a first handedness - a right-hand thread as shown. Of course, the first handedness could also be left-hand oriented in other embodiments. The exterior of cannulated screw 200 also includes a thread cutout 210, which is a helical opening extending between the exterior and cavity 202. Cutout 210 is oppositely handed from that of first thread 208 and is configured to receive a portion of blocking screw 300, as will be discussed more fully below. Although shown extending only along a portion of the length of screw 200, cutout 210 can extend along the entire length. Similarly, in other embodiments, cannulation 202 may only extend along a portion of the length of screw 200 and only a portion of the exterior of screw 200 may include first thread 208.

First thread 208 may have a pitch ranging from 3-5. In some embodiments, cannulated screw 200 may have at least two differently pitched threaded portions designed to promote compression of the fracture fragments of the bone thus increasing rotational stability. The pitched threaded portions may be two separate threads or be contained within a single continuous thread. Moreover, first thread 208 may have a major diameter of between 5-20 mm while minor diameter 212 of screw 200 may be between 3-15. As discussed below, these ranges preferably coincide with certain parameters of screw 300.

As is also shown in FIGS. 2A-2C, cannulated screw 200 includes a tip portion 214 located at distal end 206 of cannulated screw 200. Tip portion 214 includes an opening connecting cannulation 202 to the exterior of cannulated screw 200. Tip portion 214 includes a flute 216 for creating a channel and for removing debris. In other embodiments, screw 200 may include other similar cutting aids. It is to be understood that screw 200 may take on the design of any known screw, as long as such is designed to cooperate with screw 300 (e.g., including a cutout 210).

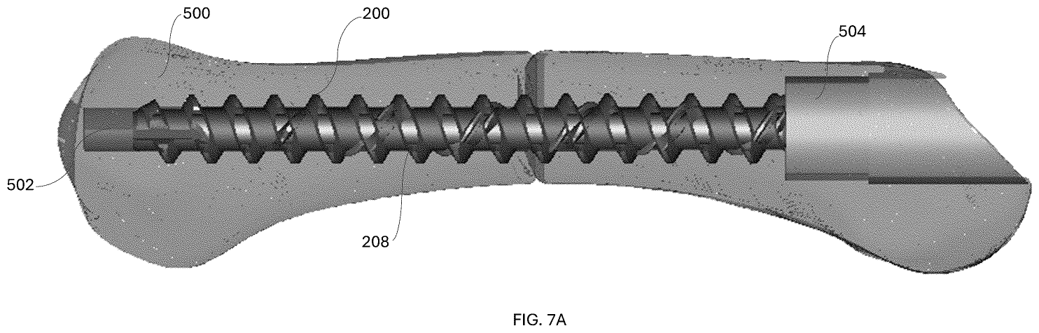

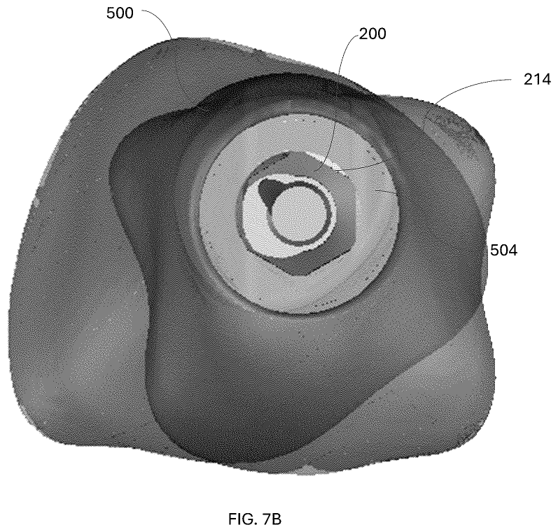

Cannulated screw 200 also includes a drive portion 218 located at proximal end 204 of cannulated screw 200 that allows cannulated screw 200 to be rotated into position with a drive tool. Drive portion 214 is shown in FIGS. 2D and 7B to be a hexagonal outer surface located at the beginning of the body of cannulated screw 200. This drive portion can be engaged by a hexagonal hollow driver to permit the rotation of the screw. In other embodiments cannulated screw 200 may include a head with drive portion 214 integrated within and could exhibit more of a traditional screw head design (e.g., a head larger than a shaft). Drive portion 214 may have a drive shape that is a slotted, a cruciform, a square, a multiple-square, a internal hex, a pentalobular, a hexalobular, a combination, a external, or a tamper resistant drive shape. As shown in FIG. 2D, cutout 210 extends through any such drive portion 214.

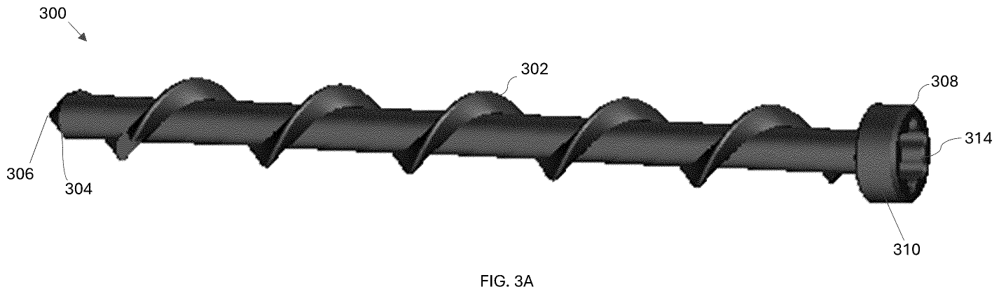

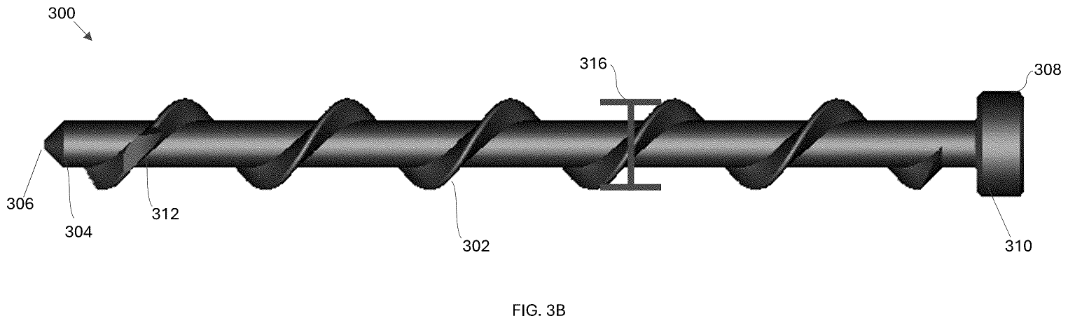

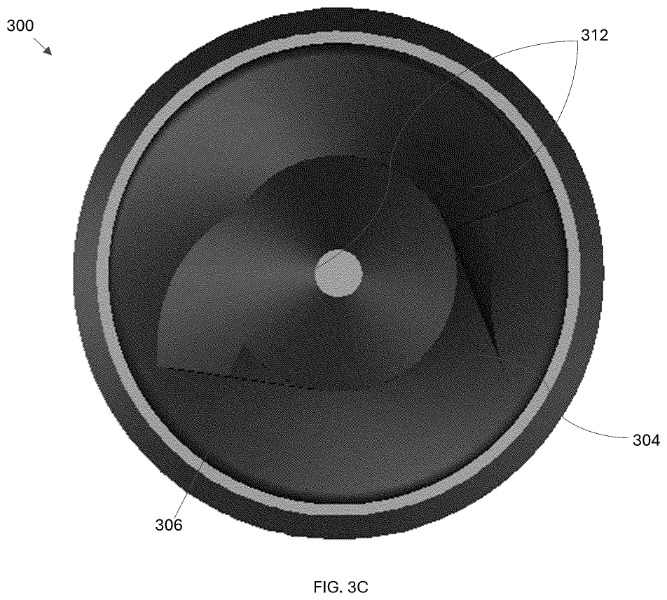

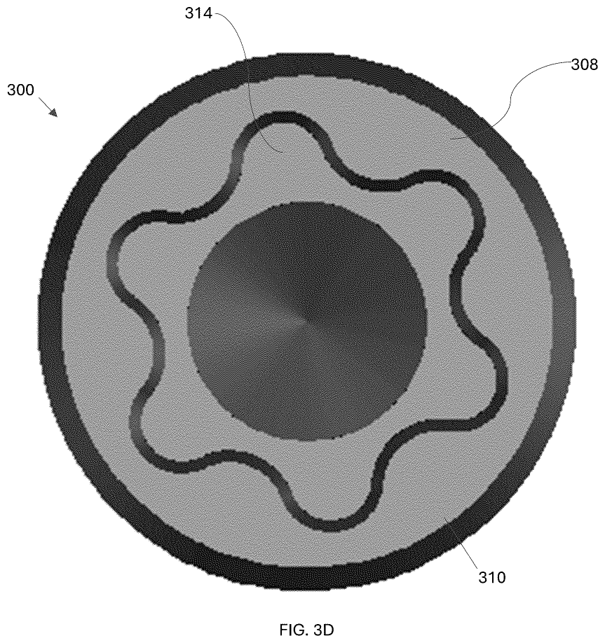

Blocking screw 300, as seen in FIGS. 3A-3D, is configured to fit within cannulated screw 200. Blocking screw 300 includes second thread 302 with a second handedness, a tip 306 at distal end 304 of the screw, and head 308 at proximal end 310 of blocking screw 300. The second handedness is in a opposite orientation from the first handedness of cannulated screw 200 (i.e., shown as a left-hand thread). Of course, this would differ if the handedness of thread 208 differed. Tip portion 306 located at distal end 304 of cannulated screw 200 includes flute 312 for creating/cutting a channel. In other embodiments, screw 300 may include other similar cutting aids, in a similar vein as is discussed above in connection with screw 200.

As best shown in FIGS. 3A, 3B, and 3D, blocking screw 300 includes drive portion 314 located at proximal end 310 of blocking screw 300 that allows blocking screw 300 to be rotated into position. In particular, head 308 includes drive portion 314 integrated within. Drive portion 314 is shown to have a hexalobular drive shape in FIG. 3D. In other embodiments the drive shape can be slotted, cruciform, square, multiple-square, internal hex, pentalobular shaped. The drive shape could also be a combination thereof, or even an external or a tamper resistant drive shape.

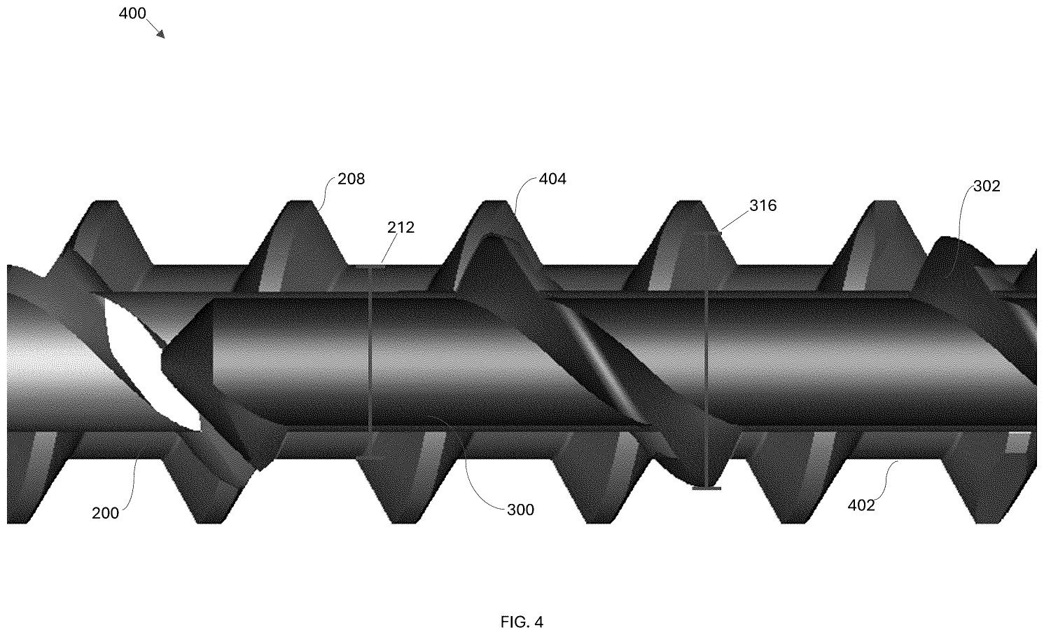

Second thread 302 may have a pitch ranging from 6-10. Unlike cannulated screw 200, blocking screw 300 includes a single pitch in order to properly engage with the cannulated screw. Moreover, second thread 208 may have a major diameter 316 of between 3-18 while the minor diameter of screw 300 may be between 2-14. Major diameter 316 of blocking screw 300 is greater than minor diameter 212 of cannulated screw 200. This is best shown in the cross-sectional view of FIG. 4, where upon placement of blocking screw 300 into cannulated screw 200 crest 404 of second thread 302 is exposed above root 402 of first thread 208 allowing second thread 302 to engage a bone.

Cutout 210 is configured to receive thread 302 of blocking screw 300 and is a helical opening between cavity 202 and the exterior of screw 200 having a handedness, pitch, and width approximately the same as thread 302. When cutout 210 intersects with first thread 208 cutout 210 extends into thread 208 creating an opening within thread 208 configured to receive at least a portion of second thread 302, as is seen in FIG. 4. In the process of rotating blocking screw 300 into cannulated screw 200 thread 302 may be rotated as to occupy the opening of cutout 210 and at least partially extends therefrom to define an exterior portion of cannulated screw 200. When blocking screw 300 is fully integrated within cannulated screw 300, second thread 302 may run across the entire length of cutout 210. Occupying cutout 210 allows thread 302 to directly contact bone 500 while minimizing the resistance attributed to thread 302 contacting screw 200.

The opposite handedness of first thread 208 and second thread 302 creates an interweaving pattern that acts to prevent assembly 100 from inadvertently dislodging from the bone. As a result of the combination of cannulated screw 200 and blocking screw 300 screw assembly 100 is rotationally and axially stable. This is includes during normal movement of the bone being repaired or even upon exterior forces such as vibrations.

In some embodiments blocking screw 300 and cannulated screw 200 may have similar thread pitches. In other embodiments blocking screw 300 and cannulated screw 200 may have different pitches. as noted above, whereby the implantation of differing pitches may act to increase the compression of a fractured bone.

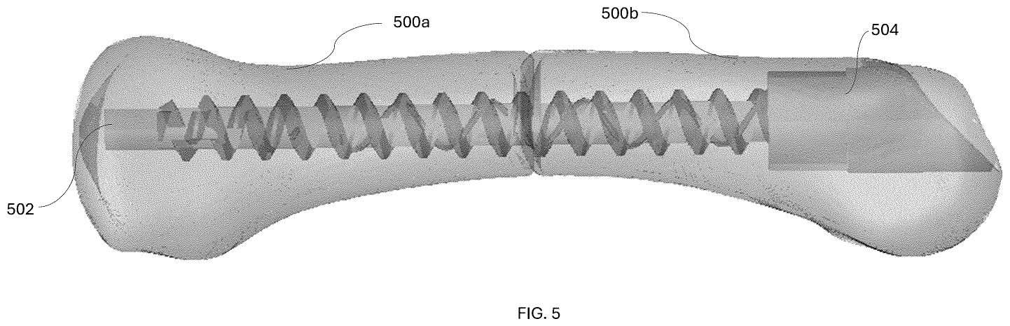

FIG. 5 illustrates a fractured bone 500 that has been prepped for the insertion of screw assembly 100. In particular a hole 502 has been drilled longitudinally across a fracture 501 and through two bone fragments 500a, 500b. Hole 502 is also shown pre-tapped, although depending upon the structures of assembly 100, such may or may not be necessary. For instance, it may only be necessary to pre-tap hole 502 to receive the larger first thread 208. Affixed in hole 502 may be a guide wire such as a K-wire (not shown) to ensure that centering is maintained through the insertion of screw assembly 100. An entry portal 504 in the form of a counterbore has also been prepared in the bone and acts to allow screw assembly 100 to be placed within fractured bone 500 without exposure to the area exterior to the bone, so as to reduce irritation and unwanted interaction with other anatomical structures.

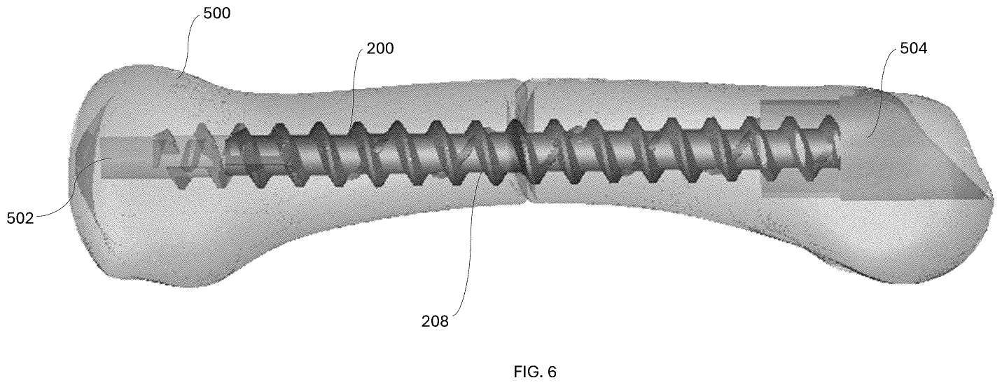

FIGS. 6-9B show the insertion of the different components of assembly 100 into fractured bone 500. Cannulated screw 200 is first rotated in a clockwise or counter-clockwise fashion into fractured bone 500 (depending upon the handedness), as shown in FIGS. 6-7A. This step can be accomplished through the use of a powered or unpowered insertion tool (not shown) that cooperates with drive portion 214 of screw 200. Any guidewire placed within the bone is preferably received in cavity 202 to guide the insertion of screw 200.

Where hole 502 was predrilled, like is shown in the drawings, cannulated screw 200 may follow predrilled hole 502 to reduce the chance of further fractures and to allow first threads 208 to directly cut into hole 502. Counterbore entry portal 504 permits cannulated screw 200 to be rotated to a position that is at least flush with the exterior of fractured bone 500 thus reducing the irritation to the area around fractured bone 500.

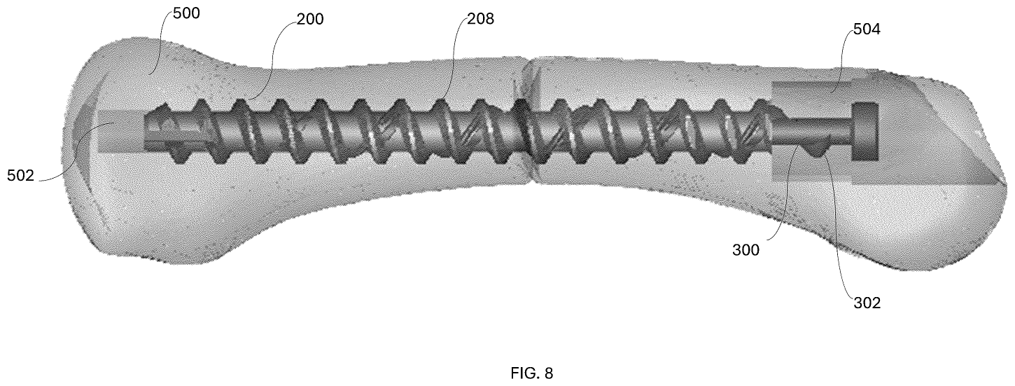

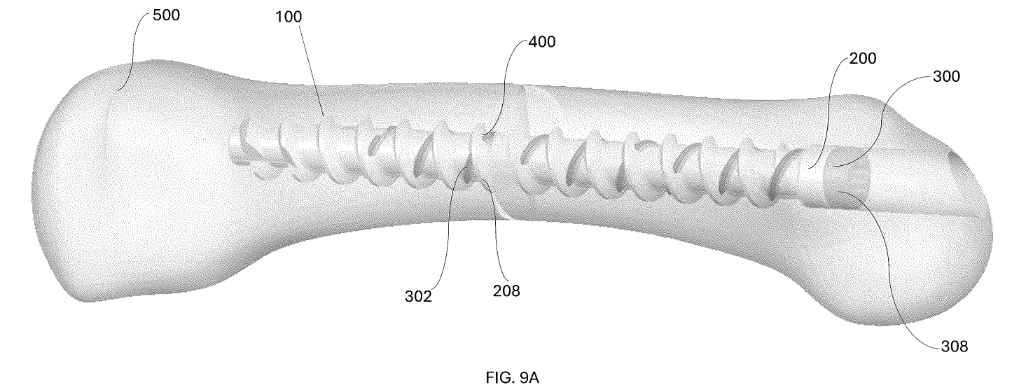

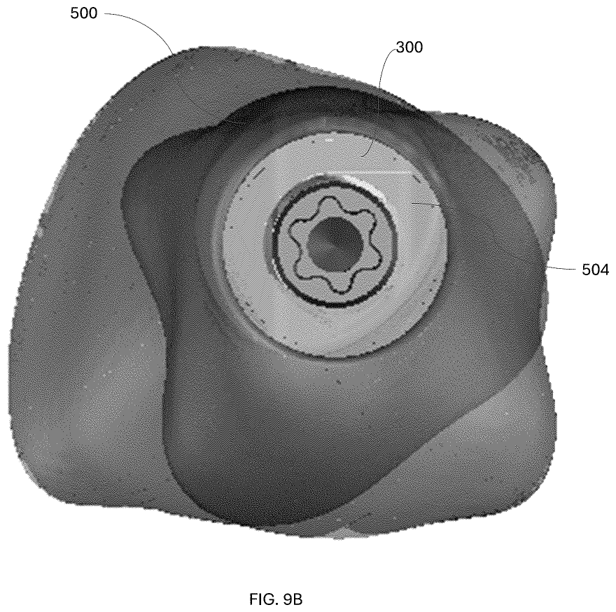

FIGS. 8, 9A and 9B show the insertion of blocking screw 300 into the already positioned cannulated screw 200. Because of its different handedness, blocking screw 300 is rotated in an opposite direction to that which screw 200 was rotated into place. Rotating blocking screw 300 into cannulated screw 200 exposes second thread 302 into bone 500 located outside of cannulated screw 200. As blocking screw 300 is rotated into place it further engages with fractured bone 500 to create a cross-working thread assembly 400 between first thread 208 and second thread 302. These screw insertion steps can be accomplished through the use of a powered or unpowered insertion tool (not shown) that cooperates with the drive portion thereof. As noted above, this results in an intertwining pattern that not only compresses the bone, but acts to lock assembly 100 into place making it difficult to come loose. It is noted that because thread 302 is smaller, pre-drilling may not be required.

FIGS. 9A and 9B illustrates screw assembly 100 fully integrated within fractured bone 500. Head 308 is in direct contact with proximal end 204 of cannulated screw 200. Threads 208 and 302 are in engagement with bone 500 thereby creating cross-working thread assembly 400. Second thread 302 intersects thread 208 due to having an orientation opposite of first thread 208. The intersection between threads 208 and 302 creates a rotational resistance in assembly 100 in both the clockwise and counterclockwise direction thus locking the assembly into place. For loosening to occur in assembly 100 an independent force must be applied to a single screw in a opposite order from the insertion order.

In cases where it is desirable or necessary to remove assembly 100, blocking screw 300 must first be rotated in a opposite direction from the insertion direction. Afterwards cannulated screw 200 may be removed by rotating screw 200 in a opposite direction from the insertion direction. These steps can be accomplished through the use of a powered or unpowered insertion tool (not shown) that cooperates with the drive portion of screw 200 and 300. Blocking screw 300 must be removed first as thread 302 prevents the rotation of cannulated screw 200 as previously explained.

Screws 200 and 300 may be made of a biocompatible material. The biocompatible material may be stainless steel, titanium, a titanium alloy, a polymer, any material not harmful to living tissue, or a combination thereof. Cannulated screw 200 may be made of the same material or a different material from that of blocking screw 300. Screws 200 and 300 may be manufactured using thread rolling, thread whirling, gun drilling, computer numerical control (CNC) cutting, or additive manufacturing. Additionally screws 200 and 300 may be heat treated in order to improve mechanical properties. After undergoing heat treatment, screws 200 and 300 may receive surface treatment to remove impurities to improve performance. Surface treatment may include acid etching, deburring, polishing, or similar treatment methods. One or more portions of assembly 100 could include porous surfaces or graft retainers to promote bone growth into and with the assembly to thereby improve upon fixation.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A screw assembly comprising:
a cannulated screw including a first thread having a first handedness; and
a blocking screw including a second thread having a second handedness opposite of the first handedness,
wherein when the blocking screw is inserted within the cannulated screw a portion of the second thread extends out of a portion of the cannulated screw.

2. The screw assembly of claim 1 wherein the cannulated screw includes a thread cutout for receiving a portion of the second thread.

3. The screw assembly of claim 1, wherein the first and second threads have different pitches.

4. The screw assembly of claim 1, wherein the first thread has at least two different pitches.

5. The screw assembly of claim 1, wherein the first thread has a first threaded section and a second threaded section having different pitches.

6. The screw assembly of claim 1, wherein a major diameter of the blocking screw is greater than a minor diameter of the cannulated screw.

7. The screw assembly of claim 1, wherein the first handedness is right-hand oriented, and the second handedness is left-hand oriented.

8. The screw assembly of claim 1, wherein the first handedness is left-hand oriented, and the second handedness is right-hand oriented.

9. The screw assembly of claim 1, wherein the blocking screw includes a head at a proximal end with a drive shape that is one of a slotted, a cruciform, a square, a multiple-square, a internal hex, a pentalobular, a hexalobular, a combination, a external, or a tamper resistant shape.

10. The screw assembly of claim 1, wherein the cannulated screw includes a head at a proximal portion of the cannulated screw.

11. The screw assembly of claim 10, wherein the head has a drive shape that is one of a slotted, a cruciform, a square, a multiple-square, a internal hex, a pentalobular, a hexalobular, a combination, a external, or a tamper resistant shape.

12. The screw assembly of claim 10, wherein the cannulated screw includes a thread cutout that extends through the head of the cannulated screw.

13. A screw assembly comprising:
a cannulated screw including a first thread having a first handedness and a thread cutout extending a length of the cannulated screw having a helical opening between a cavity and an exterior of the cannulated screw;
a blocking screw including a second thread having a second handedness opposite of the first handedness; and
wherein when the blocking screw is inserted within the cannulated screw a portion of the second thread extends partially out of the thread cutout and engages a bone.

14. The screw assembly of claim 13, wherein the first and second threads have different pitches.

15. The screw assembly of claim 13, wherein the first thread has a first threaded section and a second threaded section having different pitches.

16. The screw assembly of claim 13, wherein a major diameter of the blocking screw is greater than a minor diameter of the cannulated screw.

17. The screw assembly of claim 13, wherein the first handedness is right-hand oriented, and the second handedness is left-hand oriented.

18. The screw assembly of claim 13, wherein the blocking screw includes a head at a proximal end with a drive shape.

19. The screw assembly of claim 13, wherein the cannulated screw includes a head at a proximal portion of the cannulated screw with a drive shape.

20. The screw assembly of claim 19, wherein the cannulated screw includes a thread cutout that extends through the head of the cannulated screw.
